(19) United States
(12) Patent Application Publication
(10) Pub. No.: US 2005/0171127 A1
(43) Pub. Date: Aug. 4, 2005

(54) Pharmaceutical compositions comprising abacavir and lamivudine

(76) Inventors: Robin Currie, Durham, NC (US); Gary Wayne Goodson, Durham, NC (US)

Correspondence Address: DAVID J LEVY, CORPORATE INTELLECTUAL PROPERTY;GLAXOSMITHKLINE, FIVE MOORE DR., PO BOX 13398, RESEARCH TRIANGLE PARK, NC, 27709-3398, US

(21) Appl. No.: 10/516,577

(22) PCT Filed: Jun. 3, 2003
(86) PCT No.: PCT/US03/17347

Related U.S. Application Data
(60) Provisional application No. 60/385,717, filed on Jun. 4, 2002.

(51) Int. Cl. A61K 31/522; A61K 31/513

(52) U.S. Cl. 514/263.32; 514/269

(57) ABSTRACT

A pharmaceutical composition comprising (1S,cis)-4-[2-amino-6-(cyclopropylamino)-9H-purin-9-yl]-2-cyclopentene-1-methanol and (2R,cis)-4-amino-1-(2-hydroxymethyl-1,3-oxathiolan-5-yl)-(1H)-pyrimidin-2-one, in an amount which achieves antiviral efficacy, a process for the preparation of such a composition, and a method of inhibiting human immunodeficiency virus (HIV) which comprises administering such a composition to an HIV infected patient is disclosed.

The present Invention relates to pharmaceutical compositions combining the agents (1S,cis)-4-[2-amino-6-(cyclopropylamino)-9H-purin-9-yl]-2-cyclopentene-1-methanol and (2R,cis)-4-amino-1-(2-hydroxymethyl-1,3-oxathiolan-5-yl)-(1H)-pyrimidin-2-one into a single form, useful in the treatment of diseases in mammals, including humans.

### BACKGROUND OF THE INVENTION

(1S,cis)-4-[2-amino-6-(cyclopropylamino)-9H-purin-9-yl]-2-cyclopentene-1-methanol (also known as abacavir, 1592U89, Ziagen®) and its antiviral use, particularly against HIV infections is described in European Patent Specification Number 0434450. The succinate salt of (1S,cis)-4-[2-amino-6-(cyclopropylamino)-9H-purin-9-yl]-2-cyclopentene-1-methanol is described in WO96/06844. The hemisulfate salt of (1S, cis)-4-[2-amino-6-(cyclopropylamino)-9H-purin-9-yl]-2-cyclopentene-1-methanol is described in WO98/52949.

(2R,cis)-4-amino-1-(2-hydroxymethyl-1,3-oxathiolan-5-yl)-(1H)-pyrimidin-2-one, (also known as lamivudine, EPIVIR®, 3TC®, -cis-1-[2-(hydroxymethyl)-1,3-oxathiolan-5-yl]cytosine, (−)2′,3′-dideoxy, 3′-thiacytidine) has proven antiviral activity against human immunodeficiency virus (HIV) and other viruses such as hepatitis B. (2R,cis)-4-Amino-1-(2-hydroxymethyl-1,3-oxathiolan-5-yl)-(1H)-pyrimidin-2-one and its use against HIV are described in EP 0382526 and WO91/17159. Crystalline forms of (2R,cis)-4-amino-1-(2-hydroxymethyl-1,3-oxathiolan-5-yl)-(1H)-pyrimidin-2-one are described in WO92/21676. Combinations of (2R,cis)-4-amino-1-(2-hydroxymethyl-1,3-oxathiolan-5-yl)-(1H)-pyrimidin-2-one with other reverse transcriptase inhibitors are described in WO92/20344. The synergistic effect of the combination of (1S,cis)-4-[2-amino-6-(cyclopropylamino)-9H-purin-9-yl]-2-cyclopentene-1-methanol and (2R,cis)-4-amino-1-(2-hydroxymethyl-1,3-oxathiolan-5-yl)-(1H)-pyrimidin-2-one is described in WO96/30025.

The success of modem multiple-drug treatments for HIV often requires strict compliance with a complex treatment regimen that can require the administration of many different drugs per day, administered at precisely timed intervals with careful attention to diet. Patient non-compliance is a well known problem accompanying such complex treatment regimens. Patient non-compliance is a critical problem in the treatment of HIV because such non-compliance may lead to the emergence of multiple-drug resistant strains of HIV.

The present invention addresses the issue of non-compliance by formulating multiple active ingredients, (1S,cis)-4-[2-amino-6-(cyclopropylamino)-9H-purin-9-yl]-2-cyclopentene-1-methanol and (2R,cis)-4-amino-1-(2-hydroxymethyl-1,3-oxathiolan-5-yl)-(1H)-pyrimidin-2-one, into a single tablet. However, simply combining the two drugs into a single tablet would result in a tablet size too large to swallow without difficulty. Furthermore, the greater the amount of drug in the formulation, the more excipients are needed in order to compress the mixture into a tablet. Increased amounts of some excipients can have adverse effects on tablet properties and can lead to problems of, for example, dissolution, content uniformity, hardness, and segregation.

The amounts of (2R,cis)-4-amino-1-(2-hydroxymethyl-1,3-oxathiolan-5-yl)-(1H)-pyrimidin-2-one in combination with (1S,cis)-4-[2-amino-6-(cyclopropylamino)-9H-purin-9-yl]-2-cyclopentene-1-methanol that are required to achieve a therapeutic effect are one gram or more. At this high drug loading, it is difficult to compress a tablet to an acceptable size to administer to a patient. In order to achieve high drug loading in a tablet, the amount of traditional binders, diluents and fillers necessary to form the combination into a tablet that exhibits content uniformity, appropriate hardness and dissolution characteristics, and that remains intact during manufacture and storage would lead to an unacceptable tablet size.

We have discovered that the addition of a highly compressible carrier to (1S,cis)-4-[2-amino-6-(cyclopropylamino)-9H-purin-9-yl]-2-cyclopentene-1-methanol and (2R,cis)-4-amino-1-(2-hydroxymethyl-1,3-oxathiolan-5-yl)-(1H)-pyrimidin-2-one allows the manufacture of tablets of acceptable size for administration to a patient. Furthermore, such tablets exhibit good content uniformity, hardness and dissolution characteristics.

### BRIEF DESCRIPTION OF THE INVENTION

It is therefore a feature of the present invention to provide pharmaceutical compositions comprising the active ingredients (1S,cis)-4-[2-amino-6-(cyclopropylamino)-9H-purin-9-yl]-2-cyclopentene-1-methanol and (2R,cis)-4-amino-1-(2-hydroxymethyl-1,3-oxathiolan-5-yl)-(1H)-pyrimidin-2-one, or pharmaceutically acceptable derivatives thereof, in the form of a tablet with high drug loading, while maintaining favorable tablet properties and suitable tablet size.

A further feature of the present invention is to provide a method for using these pharmaceutical compositions.

### DETAILED DESCRIPTION OF THE INVENTION

The present invention provides pharmaceutical compositions comprising the active ingredients (1S,cis)-4-[2-amino-6-(cyclopropylamino)-9H-purin-9-yl]-2-cyclopentene-1-methanol and (2R,cis)-4-amino-1-(2-hydroxymethyl-1,3-oxathiolan-5-yl)-(1H)-pyrimidin-2-one, or pharmaceutically acceptable derivatives thereof, in the form of a tablet with high drug loading, while maintaining favorable tablet properties and suitable tablet size.

A further feature of the present invention is to provide a method for using these pharmaceutical compositions.

The present invention features a pharmaceutical composition, comprising:
i) a safe and therapeutically effective amount of (1S,cis)-4-[2-amino-6-(cyclopropylamino)-9H-purin-9-yl]-2-cyclopentene-1-methanol or a pharmaceutically acceptable derivative thereof;
ii a safe and therapeutically effective amount of (2R,cis)-4-amino-1-(2-hydroxymethyl-1,3-oxathiolan-5-yl)-(1H)-pyrimidin-2-one or a pharmaceutically acceptable derivative thereof; and
iii) a pharmaceutically acceptable highly compressible carrier.

The present invention also features pharmaceutical compositions comprising a safe and therapeutically effective amount of (1S,cis)-4-[2-amino-6-(cyclopropylamino)-9H-purin-9-yl]-2-cyclopentene-1-methanol (herein referred to as “abacavir”) or a pharmaceutically acceptable derivative thereof, a safe and therapeutically effective amount of (2R,cis)-4-amino-1-(2-hydroxymethyl-1,3-oxathiolan-5-yl)-(1H)-pyrimidin-2-one (herein referred to as “lamivudine) or a pharmaceutically acceptable derivative thereof; and a pharmaceutically acceptable highly compressible carrier wherein the composition has a volume in the range of 1.0-1.2 mL. Further pharmaceutical compositions of the present invention comprise abacavir and lamivudine as described above wherein the composition exhibits acceptable tablet hardness, for example of greater than 20 kilopounds at 25 kilonewtons of force for a 1375 mg tablet.

The phrase “safe and therapeutically effective amount,” as used herein, means a sufficient amount of a drug, compound, composition, product or pharmaceutical agent to abate or reverse or treat a malady in a human or other mammal without severely harming the tissues of the mammal to which the drug or pharmaceutical agent is administered.

The phrase “pharmaceutically acceptable derivative,” as used herein, means any pharmaceutically acceptable salt, solvate, ester, or salt of such ester, or any other compound which, upon administration to the recipient, is capable of providing (directly or indirectly) the intended active ingredient or any active metabolite or residue thereof.

The phrase “pharmaceutically acceptable derivative of abacavir” as used herein, means any pharmaceutically acceptable salt, solvate, ester, or salt of such ester, of abacavir, or any other compound which, upon administration to the recipient, is capable of providing (directly or indirectly) abacavir or any antivirally active metabolite or residue thereof. A preferred pharmaceutically acceptable derivative of abacavir is abacavir hemisulfate salt.

The phrase “pharmaceutically acceptable derivative of lamivudine” as used herein, means any pharmaceutically acceptable salt, solvate, ester, or salt of such ester, of lamivudine, or any other compound which, upon administration to the recipient, is capable of providing (directly or indirectly) lamivudine or any antivirally active metabolite or residue thereof.

The phrase “highly compressible carrier” as used herein means binder or filler that provides good tableting properties such as tablet hardness, low friability, and flow at quantities significantly lower than conventional fillers or binders such as Avicel® PH 101, Avicel® PH012, lactose, and other similar binders or fillers.

The phrase “drug loading,” as used herein, means the ratio of drug to total weight of tablet.

The pharmaceutical compositions of the present invention contain highly compressible carriers, for example, diluents, binders or fillers, for example, highly compressible microcrystalline cellulose. The advantages of highly compressible microcrystalline cellulose are low bulk density and high compressibility, superior compatibility and low friability. The use of highly compressible microcrystalline cellulose enables compaction at lower forces and results in the capability to manufacture 6 harder tablets. Furthermore, disintegration times of compositions made with highly compressible microcrystalline cellulose are faster compared to compositions made with conventional microcrystalline cellulose, for example, Avicel® PH101 and Avicel® PH102, at equivalent tablet hardness. The carrier(s) must be pharmaceutically acceptable in the sense of being compatible with the other ingredients of the formulation and not deleterious to the recipient thereof.

The compositions of the present invention employ a safe and therapeutically effective amounts of abacavir or a pharmaceutically acceptable derivative thereof, and lamivudine or a pharmaceutically acceptable derivative thereof, along with a safe and effective amount of a pharmaceutically acceptable highly compressible carrier. Highly compressible carriers may be diluents, binders, or fillers. Examples include, but are not limited to, highly compressible microcrystalline cellulose, for example, Ceolus®, ProSolv™, and Avicel®PH105 microcrystalline cellulose.

The present invention further features a pharmaceutical composition consisting essentially of abacavir, or a pharmaceutically acceptable derivative thereof, lamivudine, and Ceolus® microcrystalline cellulose.

Compositions of the present invention feature unit dosage forms, for example, tablets containing abacavir and lamivudine, wherein the tablet has a volume of less than 1.5 mL, advantageously less than 1.2 mL, or in the range of 1.0-1.3 mL, preferably about 1.1 mL. Tablets of the present invention containing abacavir and lamivudine exhibit properties that are advantageous for administration as a pharmaceutical composition. For example, tablets of the present invention may have a thickness of less than or equal to 8.6 mm, may exhibit low friability (<0.3%), for example, a friability of less than or equal to 0.1%, may exhibit a hardness of greater than 18 kilopounds for a 1375 mg tablet, and/or may exhibit a disintegration of less than or equal to 20 minutes, advantageously less than or equal to 12 minutes.

The present invention features pharmaceutical compositions as described above which are flowable, compressible, have low friability, good disintegration times, good tablet hardness, and acceptable dissolution.

The present invention also features pharmaceutical compositions comprising abacavir or a pharmaceutically acceptable derivative thereof, lamivudine or a pharmaceutically acceptable derivative thereof, and Ceolus® microcrystalline cellulose. Such compositions may have a volume of about 1.1 mL and/or exhibit a hardness of greater than 18 kilopounds and/or exhibit a disintegration of less than or equal to 20 minutes, advantageously less than or equal to 12 minutes.

The present invention features a pharmaceutical composition comprising abacavir, or a pharmaceutically acceptable derivative thereof and lamivudine, or a pharmaceutically acceptable derivative thereof, in an amount from about 20% to 80% of total compression weight or from about 30% to about 70% of total composition weight. The pharmaceutical composition may advantageously be in the form of a tablet, said tablet having 20%-80% drug loading or 30% to 60% drug loading, advantageously 40% to 60% drug loading.

Preferably, lamivudine is provided substantially free of the corresponding (+)-enantiomer. “Substantially free” as used herein, means that there is less than about 10% w/w of the (+)-enantiomer present compared with the amount of lamivudine. Preferably there is less than about 5% w/w of the (+)-enantiomer present compared with the amount of lamivudine.

Another feature of the present invention is to simplify treatment regimens for HIV and other viruses with the goal of enhancing patient compliance by providing a simplified dosage form containing pharmaceutically acceptable amounts of abacavir and lamivudine or pharmaceutically acceptable derivatives thereof.

The present invention also features a method for treating, reversing, reducing or inhibiting retroviral infections in particular HIV infections, in a mammal, in particular a human, which method comprises administering to said mammal a safe and effective amount of a composition according to the invention.

The present invention provides the combined use of abacavir, or a pharmaceutically acceptable derivative thereof, lamivudine, or a pharmaceutically acceptable derivative thereof and a pharmaceutically acceptable highly compressible carrier in the manufacture of a medicament for the treatment of a retroviral infection, in particular an HIV infection.

It will be appreciated by those skilled in the art that reference herein to “treatment” extends to both the prophylaxis and the treatment of an established malady, infection or its symptoms.

The compositions of the present invention may optionally employ a safe and effective amount of a diluent, a safe and effective amount of a disintegrant, and a safe and effective amount of a lubricant or any other safe and effective amounts of excipients commonly used in the art.

The compositions of the present invention may include from 0 to about 2% magnesium stearate; from about 0.05 to about 5% glidant; from 0 to about 5% sodium starch glycollate; and from about 20 to about 50% microcrystalline cellulose.

The pharmaceutical compositions of the present invention may optionally contain silicon dioxide (SiO₂), also referred to as colloidal silica, fumed silicon dioxide, fumed silica, light anhydrous silicic acid, silicic anhydride, AEROSIL™ or CAB-O-SIL™; asbestos free talc, sodium aluminosilicate, calcium silicate, powdered cellulose, microcrystalline cellulose, corn starch, sodium benzoate, calcium carbonate, magnesium carbonate, metallic stearates, calcium stearate, magnesium stearate, zinc stearate, stearowet C, starch, starch 1500, magnesium lauryl sulfate, magnesium oxide, colloidal silicon dioxide in combination with microcrystalline cellulose or ProSolv™.

Abacavir may be prepared by the method described in European Patent Specification Number 0434450 or WO95/21161, which are incorporated herein by reference hereto. The succinate salt of 1592U89 may be prepared by the method described in WO96/06844, which is incorporated herein by reference hereto. The hemisulfate salt of 1592U89 may be prepared by the method described in WO98/52949, which is incorporated herein by reference hereto. Preferred salts of abacavir include the succinate salt and the hemisulfate salt.

Methods for the preparation of lamivudine are described in, inter alia, WO 91/17159, WO 92/21676, WO 92/20669, WO 95/29174, incorporated herein by reference.

The invention is preferably presented as a pharmaceutical composition suitable for oral administration. Such compositions may conveniently be presented as discrete units such as tablets, caplets, capsules, or any other form suitable for oral administration and compatible with the compositions of the present invention, each containing a predetermined amount of the active ingredients. A particularly suitable composition may be prepared from direct compression or granulation processes. Such compositions may contain safe and effective amounts of conventional excipients such as binding agents, fillers, lubricants, or disintegrants. The tablets may also be coated according to any method known to persons skilled in the art that would not interfere with the tablets' release properties, or the other physical or chemical characteristics of the present Invention. Tablet coating is further described and delineated by Remington, The Science & Practice of Pharmacy 19th ed. 1995 incorporated herein by reference. When desired, the above formulations may also be modified by any method known to persons skilled in the art to achieve sustained release of active ingredients. The compositions may also include a safe and effective amount of other active ingredients, such as antimicrobial agents or preservatives.

These compositions of the present invention are suitable for administration to humans or other mammals particularly via an oral route of administration. However, other routes as utilised by medical practitioners and others skilled in the art of pharmaceutical dosage administration such as pharmacists and nurses are not foreclosed.

It will be appreciated by those skilled in the art that the amount of active ingredients required for use in treatment will vary according to a variety of factors, including the nature of the condition being treated and the age and condition of the patient, and will ultimately be at the discretion of the attending physician, veterinarian or health care practitioner.

In general, however, a suitable dose of abacavir for administration to a human for treatment of an HIV injection may be in the range of 0.1 to 120 mg per kilogram body weight of the recipient per day, preferably in the range of 3 to 90 mg per kilogram body weight per day and most preferably in the range 5 to 60 mg per kilogram body weight per day.

The current recommended oral dose of lamivudine for adults and adolescents is 150 mg twice daily administered in combination with other antiretroviral agents. For adults with low body weights (less than 50 kg or 10 lb.) the current recommended oral dose of lamivudine is 2 mg/kg twice daily administered in combination with other antiretroviral agents. The recommended oral dose of lamivudine in paediatric patients 3 months to 12 years of age is 4 mg/kg twice daily, up to a maximum of 150 mg twice daily administered in combination with other antiretroviral agents.

Compositions of the present invention enable patients greater freedom from multiple dosage medication regimens and ease the needed diligence required in remembering complex daily dosing times and schedules. By combining abacavir and lamivudine into a single dosage form, the desired daily doses may be presented in a single dose or as divided doses, administered at appropriate intervals, for example as two, three, four or more sub-doses per day. The compositions of the present invention are particularly suitable for administration as a single dose once daily. Advantageously, the compositions of the present invention may be administered once daily.

The compositions of the present invention conveniently allow administration of two separate compounds in unit dosage form containing, for example, from about 15 to about 1200 mg of abacavir, particularly from about 100 to about 750 mg of abacavir, and most particularly about 700 mg of abacavir, from about 15 to about 1000 mg of lamivudine, particularly from about 100 to about 500 mg of lamivudine and most particularly 300 mg of lamivudine per unit dosage form. The composition of the present invention may be used in combination with other pharmaceutical formulations as a component of a multiple drug treatment regimen.

Compositions of the present invention may also be packaged as articles of manufacture comprising a safe and therapeutically effective amount of abacavir, or a pharmaceutically acceptable derivative thereof; and a safe and therapeutically effective amount of lamivudine, or a pharmaceutically acceptable derivative thereof and a safe and effective amount of a pharmaceutically acceptable highly compressible carrier.

Any of the various methods known by persons skilled in the art for packaging tablets, caplets, or other solid dosage forms suitable for oral administration, that will not degrade the components of the present invention, are suitable for use in packaging. Tablets, caplets, or other solid dosage forms suitable for oral administration, may be packaged and contained in various packaging materials particularly glass and plastic bottles and also including unit dose blister packaging. The packaging material may also have labelling and information related to the pharmaceutical composition printed thereon. Additionally, an article of manufacture may contain a brochure, report, notice, pamphlet, or leaflet containing product information. This form of pharmaceutical information is referred to in the pharmaceutical industry as a “package insert.” A package insert may be attached to or included with a pharmaceutical article of manufacture. The package insert and any article of manufacture labelling provides information relating to the pharmaceutical composition. The information and labelling provides various forms of information utilised by health-care professionals and patients, describing the composition, its dosage and various other parameters required by regulatory agencies such as the United States Food and Drug Agencies.

The compositions of the present invention can be formulated using methods and techniques suitable for the compositions physical and chemical characteristics and that are commonly employed by persons skilled in the art in preparing oral dosage forms utilising direct compression or granulation processes. Remington, The Science & Practice of Pharmacy, p. 1615-1623, 1625-1648, and other applicable sections, 19th ed. (1995).

Compositions of the present Invention in their method aspect are administered to a human or other mammal in a safe and effective amount as described herein. These safe and effective amounts will vary according to the type and size of mammal being treated and the desired results of the treatment.

### EXAMPLES

The following examples further describe and demonstrate particular embodiments within the scope of the present Invention. The examples are given solely for illustration and are not to be construed as limitations as many variations are possible without departing from spirit and scope of the Invention.

### Example 1

Dual Combination Tablet Containing Abacavir and Lamivudine

| | Quantity | Quantity |
|---|---|---|
| Component | (mg/tablet) | (% w/w) |
| Abacavir Hemisulfate | 702.0 | 51.05 |
| Lamivudine | 300.0 | 21.82 |
| Ceolus ® | 309.06 | 22.48 |
| Sodium Starch Glycolate | 55.0 | 4.00 |
| Magnesium Stearate | 8.94 | 0.65 |
| Total Tablet Weight | 1375.0 | |

Bulk Preparation Method

The quantities of the present example of manufacturing procedure are based on a typical batch size of 300 kg and may be adjusted depending on batch size.

First the components are weighed from bulk containers in the following amounts:

| Ingredients | Amount (kg) |
|---|---|
| Abacavir hemisulfate | 153.2 |
| Lamivudine | 65.5 |
| Ceolus ® (Microcrystalline Cellulose, NF) | 67.3 |
| Sodium Starch Glycolate | 12.0 |
| Magnesium Stearate | 2.0 |

The components are then sieved using a Russel-SIV equipped with a 14 mesh (1.4 mm opening) or an equivalent sieve and mesh, and deposited into a stainless-steel blending container.

The abacavir, lamivudine, Ceolus®, and sodium starch glycolate, NF are blended for 12 minutes using a suitable blender, such as a Matcon-Buls bin-type blender, a V-blender or equivalent. The magnesium stearate is then added to the mixture and blending is continued for approximately 2 minutes.

The lubricated blend is then compressed using a suitable rotary tablet press, typically a Fette 2090 or equivalent. In-process controls for tablet weight and hardness are applied at appropriate intervals throughout the compression run and adjustments to the tablet press are made as necessary.

### Example 2

Comparative Batch Data for Different Carriers/Binders

Tablets were weighed on an analytical balance. A digital caliper was used to measure the thickness of the tablets. Tablet hardness was measured on a suitable hardness tester by placing the tablets lengthwise between the crushing jaws. Powder flow was determined by placing a powder sample into a Flodex™. The sample was then allowed to sit undisturbed for fifteen seconds prior to being discharged through a stainless steel orifice. The orifices were changed as needed until the smallest size was determined that allowed the powder to flow freely. Friability and disintegration was measured according to the current U.S. Pharmacopeia (USP 25-NF 20).

| | | Avicel | Avicel | |
|---|---|---|---|---|
| Test | Ceolus | PH101 | PH105 | Prosolv |
| Compression Weight | 1000 | 1000 | 1000 | 1000 |
| (mg) | | | | |
| Thickness (mm) | 6.07 | 6.10 | 6.06 | 6.23 |
| Friability (%) | 0.07 | 2.6 | 0.24 | 0.24 |
| Hardness (kp) | 19.3 | 13.1 | 15.0 | 17.4 |
| Disintegration (min) | 10.95 | 4.33 | 15.14 | not tested |
| Flow (mm) | 20.5 | 20 | 23.5 | 21 |

Note:
Theoretical tablet weights equivalent to 1375 mg; however due to laboratory tooling selection all batches were compressed at 1000 mg with identical tooling.

Acceptable attributes based on data above:

| | | Avicel ® | Avicel ® | |
|---|---|---|---|---|
| Test | Ceolus ® | PH101 | PH105 | Prosolv ™ |
| Friability | yes | no | marginal | marginal |
| Hardness | yes | no | marginal | yes |
| Disintegration | yes | yes | no | not tested |
| Flow | yes | yes | no | yes |
| Appearance | yes | no | yes | no |

The application of which this description and claims form part may be used as a basis for priority in respect of any subsequent application. The claims of such subsequent application may be directed to any feature or combination of features described herein. They may take the form of product, composition, process or use claims and may include, by way of example and without limitation, one or more of the following claims.

1. A pharmaceutical composition comprising:
i) a safe and therapeutically effective amount of (1S,cis)-4-[2-amino-6-(cyclopropylamino)-9H-purin-9-yl]-2-cyclopentene-1-methanol or a pharmaceutically acceptable derivative thereof;
ii) a safe and therapeutically effective amount of (2R,cis)-4-amino-1-(2-hydroxymethyl-1,3-oxathiolan-5-yl)-(1H)-pyrimidin-2-one or a pharmaceutically acceptable derivative thereof; and
iii) a pharmaceutically acceptable highly compressible carrier.

2. A pharmaceutical composition comprising:
i) a safe and therapeutically effective amount of (1S,cis)-4-[2-amino-6-(cyclopropylamino)-9H-purin-9-yl]-2-cyclopentene-1-methanol or a pharmaceutically acceptable derivative thereof;
ii) a safe and therapeutically effective amount of (2R,cis)-4-amino-1-(2-hydroxymethyl-1,3-oxathiolan-5-yl)-(1H)-pyrimidin-2-one or a pharmaceutically acceptable derivative thereof; and
iii) a pharmaceutically acceptable highly compressible carrier
wherein said composition has a volume in the range of 1.0-1.3 mL.

3. The present invention features a pharmaceutical composition in tablet form comprising:
i) a safe and therapeutically effective amount of (1S,cis)-4-[2-amino-6-(cyclopropylamino)-9H-purin-9-yl]-2-cyclopentene-1-methanol or a pharmaceutically acceptable derivative thereof;
ii) a safe and therapeutically effective amount of (2R,cis)-4-amino-1-(2-hydroxymethyl-1,3-oxathiolan-5-yl)-(1H)-pyrimidin-2-one or a pharmaceutically acceptable derivative thereof; and
iii) a pharmaceutically acceptable highly compressible carrier
wherein said composition exhibits a tablet hardness of greater than 18 kilopounds at 25 kilonewtons of force.

4. A pharmaceutical composition according to, claim 2 wherein the pharmaceutically acceptable highly compressible carrier is selected from a group consisting of diluents, binders, and fillers.

5. A pharmaceutical composition according to claim 4 wherein the pharmaceutically acceptable highly compressible binder is selected from the group consisting of highly compressible microcrystalline cellulose.

6. A pharmaceutical composition according to claim 5 wherein the compressible microcrystalline cellulose is Ceolus® microcrystalline cellulose.

7. A pharmaceutical composition according to claim 2 comprising (1S,cis)-4-[2-amino-6-(cyclopropylamino)-9H-purin-9-yl]-2-cyclopentene-1-methanol, or a pharmaceutically acceptable derivative thereof, (2R,cis)-4-amino-1-(2-hydroxymethyl-1,3-oxathiolan-5-yl)-(1H)-pyrimidin-2-one, or a pharmaceutically acceptable derivative thereof, wherein said (1S,cis)-4-[2-amino-6-(cyclopropylamino)-9H-purin-9-yl]-2-cyclopentene-1-methanol and (2R,cis)₄-amino-1-(2-hydroxymethyl-1,3-oxathiolan-5-yl)-(1H)-pyrimidin-2-one are present in an amount of 20% to 80% of total composition weight.

8. A pharmaceutical composition according to claim 2 wherein the amount of (1S,cis)-4-[2-amino-6-(cyclopropylamino)-9H-purin-9-yl]-2-cyclopentene-1-methanol is from about 15 to about 1200 mg per unit dosage form.

9. A pharmaceutical composition according to claim 2 wherein the amount of (2R,cis)-4-amino-1-(2-hydroxymethyl-1,3-oxathiolan-5-yl)-(1H)-pyrimidin-2-one is from about 15 to about 1500 mg per unit dosage form.

10. A pharmaceutical composition according to claim 9 wherein the amount of (2R,cis)-4-amino-1-(2-hydroxymethyl-1,3-oxathiolan-5-yl)-(1H)-pyrimidin-2-one is from about 100 to about 500 mg per unit dosage form.

11. A pharmaceutical composition according to claim 10 wherein the amount of (2R,cis)-4-amino-1-(2-hydroxymethyl-1,3-oxathiolan-5-yl)-(1H)-pyrimidin-2-one is 300 mg per unit dosage form.

12. The pharmaceutical composition according to claim 2 wherein (2R,cis)-4-amino-1-(2-hydroxymethyl-1,3-oxathiolan-5-yl)-(1H)-pyrimidin-2-one is provided substantially free of the corresponding (+)-enantiomer.

13. The pharmaceutical composition according to claim 2 wherein (2R,cis)-4-amino-1-(2-hydroxymethyl-1,3-oxathiolan-5-yl)-(1H)-pyrimidin-2-one is provided such that the corresponding (+)-enantiomer is present in an amount of not more than about 5% w/w of the amount of lamivudine.

14. The pharmaceutical composition according to claim 2 wherein the pharmaceutically acceptable derivative of (1S,cis)-4-[2-amino-6-(cyclopropylamino)-9H-purin-9-yl]-2-cyclopentene-1-methanol is the hemisulfate salt.

15. The pharmaceutical composition according to claim 2 wherein the pharmaceutically acceptable highly compressible carrier is present in an amount of 5% to about 50% by weight.

16. A pharmaceutical composition comprising (1S,cis)-4-[2-amino-6-(cyclopropylamino)-9H-purin-9-yl]-2-cyclopentene-1-methanol, or a pharmaceutically acceptable derivative thereof, (2R,cis)-4-amino-1-(2-hydroxymethyl-1,3-oxathiolan-5-yl)-(1H)-pyrimidin-2-one, and Ceolus® microcrystalline cellulose.

17. A pharmaceutical composition consisting essentially of (1S, cis)-4-[2-amino-6-(cyclopropylamino)-9H-purin-9-yl]-2-cyclopentene-1-methanol, or a pharmaceutically acceptable derivative thereof, (2R,cis)-4-amino-1-(2-hydroxymethyl-1,3-oxathiolan-5-yl)-(1H)-pyrimidin-2-one, and Ceolus® microcrystalline cellulose.

18. A pharmaceutical composition according to claim 17 wherein the pharmaceutically acceptable derivative of (1S,cis)-4-[2-amino-6-(cyclopropylamino)-9H-purin-9-yl]-2-cyclopentene-1-methanol is the hemisulfate salt.

19. A pharmaceutical composition according to claim 17 wherein the composition has a has a volume in the range of 1.0-1.3 mL.

20. A pharmaceutical composition according to claim 2 in the form of a tablet.

21. A pharmaceutical composition according to claim 2 for once daily administration.

22. A pharmaceutical composition according to claim 20 wherein the composition is coated with a pharmaceutically acceptable coating.

23. (canceled)

24. (canceled)

25. A method for treating, reversing, reducing or inhibiting retroviral infections by administering a safe and effective amount of a composition according to claim 2.

26. The method for treating, reversing, reducing or inhibiting retroviral infections according to claim 25, wherein the retrovirus is HIV.

27. (canceled)

28. (canceled)

29. (canceled)

30. (canceled)

31. (canceled)
